(19) United States
(12) Patent Application Publication
(10) Pub. No.: US 2016/0136646 A1
(43) Pub. Date: May 19, 2016

(54) Interconnect Adaptor

(71) Applicant: PRESIDENT AND FELLOWS OF HARVARD COLLEGE, Cambridge, MA (US)

(72) Inventors: Donald E. Ingber, Boston, MA (US); Christopher David Hinojosa, Cambridge, MA (US); Daniel Levner, Cambridge, MA (US); Josh Isaac Nielsen Resnikoff, Somerville, MA (US); Guy Thompson, III, Lexington, MA (US)

(21) Appl. No.: 14/899,106

(22) PCT Filed: Jun. 26, 2014
(86) PCT No.: PCT/US14/44417

§ 371 Date: Dec. 16, 2015

Related U.S. Application Data
(60) Provisional application No. 61/839,702, filed on Jun. 26, 2013.

(51) Int. Cl. B01L 3/00; C12M 1/00; C12M 3/06

(52) U.S. Cl. CPC B01L 3/563; B01L 3/502715; C12M 23/16; C12M 29/00; B01L 2200/027; B01L 2200/025; B01L 2200/0689; B01L 2300/12; B01L 2200/10; B01L 2300/0861; B01L 2300/0887; B01L 2300/123; B01L 2200/0684

(57) ABSTRACT

An interconnect adaptor for connecting a microfluidic device to a fluidic system. The interconnect adapter includes a base substrate and a nozzle array. The base substrate includes a first side and a second side. The nozzle array includes two or more nozzles extending away from the base substrate. Each nozzle includes an opening with a channel extending therefrom. The channels are configured to transport fluid between the microfluidic device and the fluidic system. Each of the nozzles is configured to be inserted into a respective hole in the microfluidic device, in some embodiments, the insertion forms a radially sealed connection between each nozzle and respective hole when the nozzles are inserted a predetermined distance into the respective holes.

### CROSS-REFERENCE TO RELATED APPLICATIONS

This application claims the benefit of U.S. Provisional Patent Application No. 61/839,702, filed Jun. 26, 2013, which is incorporated herein by reference in its entirety.

### GOVERNMENT SUPPORT

This invention was made with government support under grant no. W911NF-12-2-0036 awarded by U.S. Department of Defense, Advanced Research Projects Agency. The government has certain rights in the invention.

### TECHNICAL FIELD

The present invention relates to an interconnect adaptor for connecting fluidic and microfluidic devices. Specifically, the invention relates to an interconnect adaptor for interconnecting components of fluid and microfluidic systems.

### BACKGROUND

Making a robust fluid connection to organ-chips and microfluidic chips in general is critical for their successful use. In the case of organ-chips, for example, improper fluidic connection can result in insufficient media perfusion to cells in the device, introduction of air bubbles and contaminants, leaking of fluid out of the assembly, or erroneous plugging-up of fluidic inlets or outlets. In the lab setting, fluidic connection to organ-chips and microfluidic chips is often done by manually inserting metal tubes into the chip's inlet and outlet ports, and then optionally applying epoxy to their bases. Another common method used in the lab setting is to three slightly oversized tubes into the chip's ports. These processes are manually laborious, messy, and not robust.

A cartridge is an adaptor that facilitates the connection of a microfluidic chip to tubes or other fluidic conduits. Optionally, the cartridge includes elements that facilitate pumping, bubble trapping, and machine-connection. However, connecting the cartridge to the microfluidic chip remains manually laborious, messy, and not robust.

### SUMMARY

The functionality of different tissue types and organs can be implemented in a microfluidic device or “chip” that enables researchers to study these various tissue types and organs outside of the body while mimicking much of the stimuli and environment that the tissue is exposed to in-vivo. In order to facilitate this research, it is desirable to implement these microfluidic devices into interconnected components that can be easily inserted and removed from au underlying fluidic system that connects to these devices.

Microfluidic devices typically consist of numerous fluid channels that can be connected to external pumps, reservoirs, and other microscale and macroscale technology components. Where the microfluidic devices include, for example, a microfluidic organ-on-a-chip, it is desirable that these connections are reliable, have low dead volume, not leak when the connections are engaged or disengaged, withstand high fluid pressure, not introduce bubbles during operation or during engagement/disengagement, and be easy for the user to engage.

The present invention is directed to an interconnect adaptor that can be used as an interface to interconnect fluidic and microfluidic devices and/or one or more organ-on-a-chip devices to become part of a larger system. In these larger fluidic and microfluidic systems, each device can have many connections and therefore it is desirable to facilitate as many connections as possible with the device. In accordance with some embodiments of the invention, the interconnect adaptor can be configured into an array that provides two or more separate interconnections.

In some embodiments, the interconnect adaptor can include a base substrate having a front-side. A nozzle array including two or more nozzles is disposed on the front-side of the base substrate. Each nozzle of the nozzle array aligns with a hole, opening, or port (inlet or outlet) of a channel of a microfluidic device. Each nozzle includes a hole connected to an opening on the base substrate or a fluidic channel within the base substrate. In some embodiments, the hole can traverse the substrate. In some embodiments, the through hole can be connected to the opening via a channel, e.g., a microfluidic channel.

The nozzle array can be used to interconnect with an array of inlets and outlets of different channels of a microfluidic device to fluidic circuit(s) on, for example, a fluidic system or a cartridge. Similarly, an array of correspondingly aligned openings on the base substrate can be used to interconnect with an array of inlets and outlets of different channels of the cartridge to channels of a microfluidic device.

In some embodiments, the nozzles cart be inserted into the inlet or outlet of the microfluidic device channel to connect the channel to a cartridge channel. The nozzle, before insertion into the inlet or outlet, can be larger in diameter than a greatest dimension of the inlet or outlet opening. Without wishing to be bound by a theory, the nozzle can become radially compressed as it is inserted into the hole, or radially compress the chip. The radial compression, which can be determined as a function of outer diameter of the nozzle, the inner diameter of hole and the elasticity of the materials, can be selected to improve the sealing properties of the nozzle based interconnect system. The microfluidic device and the adaptor can be attached by the radial compression or may still need other mechanism for fastening such as screws, bolts, pins or clamps. Accordingly, the attached microfluidic device's weight can be supported by the radial compression of the nozzles or may still need other mechanism for fastening the microfluidic device to the adaptor.

In some embodiments, the base substrate can be attached by physical, mechanical, or chemical methods to a cartridge. For example, the base substrate can be fastened by screws, bolts, pins, clamps, or the like to the cartridge. In some embodiments, the based substrate can be bonded (e.g., glued) with the cartridge. In some embodiments, the base substrate can be “trapped” by the cartridge. For example, the base substrate of the adaptor can be sandwiched between two layers of the cartridge. In some embodiments, the base substrate can be part of the cartridge. In some embodiments, the nozzles can extend directly from holes in the cartridge without use of a base substrate.

In some embodiments, the nozzles can be arranged in a predetermined pattern on the base substrate, wherein the pattern corresponds to an array of inlets and outlets in a microfluidic device.

In some embodiments, the openings on the opposing side of the base substrate can be arranged in a predetermined pattern on the base substrate, wherein the pattern corresponds to an array of inlets and outlets in a cartridge.

In some embodiments, the adaptor can comprise a nozzle array having two or more nozzles located on the base substrate, for example the back-side. Each nozzle aligns with an inlet or outlet of a channel of a cartridge. Each nozzle having a through hole connected to a nozzle on the front-side of the substrate.

In some embodiments, an interconnect adaptor for connecting a micro:fluidic device to a fluidic system, includes abuse substrate and a nozzle array. The base substrate includes a first side. The nozzle array includes two or more nozzles. The nozzle array is located on the first side of the base substrate. The two or more nozzles extend away from the base substrate. Each of the nozzles includes an opening with a channel extending therefrom. The channels are configured to transport fluid between the microfluidic device and the fluidic system. Each of the nozzles is configured to be inserted into a respective hole in the microfluidic device. The insertion forms a radially sealed connection between each nozzle and respective hole when the nozzles are inserted into the respective holes.

In some embodiments, an interconnect adaptor for connecting a fluidic system to a compatible microfluidic device includes abase substrate and a nozzle array. The base substrate includes a first side. The nozzle array includes two or more nozzles. The nozzle array is located on the first side of the base substrate. The two or more nozzles extend away from the base substrate. Each of the nozzles includes an opening with a channel extending therefrom. The channels are configured to transport fluid between the compatible microfluidic device and the fluidic system. Each of the nozzles is configured to be inserted into a respective hole in the compatible microfluidic device. The nozzles of the nozzle array form a lock-and-key arrangement such that the nozzles can be inserted into the respective holes of only microfluidic devices that satisfy a predetermined criterion.

In some embodiments, an interconnect adapter for connecting a microfluidic device to a fluidic system includes a first portion and a second portion. The first portion includes a first substrate and a first nozzle array of two or more device-nozzles. The first substrate includes a first side and a second side. The device-nozzles are disposed on the first side of the first substrate. The two or more device-nozzles extend away from the first substrate. Each of the device-nozzles is configured to be inserted into a respective hole in the microfluidic device. Each of the device-nozzles includes a first opening. The second portion includes a second substrate and a second nozzle array of two or more cartridge-nozzles. The second substrate includes a third side and a fourth side. The cartridge-nozzles are disposed on the fourth side of the second substrate. The two or more cartridge-nozzles extend away from the second substrate. Each of the cartridge-nozzles is configured to be inserted into a respective hole in the fluidic system. Each of the cartridge-nozzles includes a second opening. The second openings are operatively coupled to respective first openings to provide for fluid flow between the microfluidic device and the fluidic system. The first portion and second portion are disposed such that the second side is proximal the third side and distal the fourth side, and such that the third side is proximal the second side and distal the first side.

These and other capabilities of the invention, along with the invention itself, will be more fully understood after a review of the following figures, detailed description, and claims.

### BRIEF DESCRIPTION OF THE DRAWINGS

The accompanying drawings, which are incorporated into this specification, illustrate one or more exemplary embodiments of the inventions and, together with the detailed description, serve to explain the principles and applications of these inventions. The drawings and detailed description are illustrative, not limiting, and can be adapted without departing from the spirit and scope of the inventions.

FIG. 1A shows a top view of the interconnect adaptor.

FIG. 1B shows a side view of the interconnect adaptor of FIG. 1A.

FIG. 1C shows the interconnect adaptor connected to an organ-chip. As shown six posts connect to the organ-chip input/outputs.

FIG. 2 shows a diagrammatic view of an organ-chip attached to a cartridge via an interconnect adaptor.

FIG. 3A shows a first perspective view of an interconnect adaptor.

FIG. 3B shows a second perspective view of the interconnect adaptor of FIG. 3A.

FIG. 4 shows an interconnect adaptor.

FIG. 5 shows an interconnect adaptor and cartridge.

FIG. 6 shows an interconnect adaptor captured by a cartridge.

FIG. 7 shows an interconnect adaptor.

FIG. 8 shows an interconnect adaptor.

FIG. 9A shows a blunt nozzle at a connection point.

FIG. 99 shows a sharpened nozzle at a connection point.

FIG. 10 shows a cartridge and interconnect adapter.

FIG. 11 shows an interconnect adapter.

### DETAILED DESCRIPTION

The present invention is directed to methods and systems for interconnecting fluidic and microfluidic devices having multiple fluid connection points with fluid sources and instruments. The fluid sources can include any liquid or gas source, such as media. The instruments can include any instruments used in fluidic and microfluidic systems, such as pumps, testing arrays having a plurality of similar devices, testing systems formed by interconnecting different devices, or analyzing devices. Interconnect adaptors disclosed herein provide art array of connection points that enable a practitioner to form multiple, simultaneous connections to the fluidic or microfluidic device(s).

Beneficially, the interconnect adaptors disclosed herein provide for connections that are simple to perform by the practitioner, can be made without seriously disrupting delicate features such as structures or cells seeded on the chip, reduce both fluid leakage and contamination, and can be made in a single motion. In some embodiments, the interconnect adaptors also maintain connections with microfluidic devices and/or cartridges using radial compression, allowing for the microfluidic devices and/or cartridges to be secured for use without additional securing mechanisms. Beneficially, this allows for mounting the microfluidic device while reducing the likelihood that the device or features thereof will be damaged or deformed due to compression against the cartridge by the additional securing mechanisms. In some embodiments, the interconnect adaptors provide reversible “snap-in, snap-out” connections that allow for easy loading and removal of microfluidic devices from the system.

In some embodiments, a system 100 includes a cartridge 600, a microfluidic device 500, and an interconnect adaptor 300. In some embodiments, the interconnect adaptor disclosed herein can be used in fluidic and microfluidic systems such as those described in PCT Application No. PCT/US2012/068725, filed Dec. 10, 2012, and PCT Application No. PCT/US2012/068766, filed Dec. 10, 2012, each of which is hereby incorporated by reference in its entirety.

The cartridge 600 is configured to hold at least one microfluidic device 500 hereon. The cartridge 600 includes a plurality of fluidic channels 720 therethrough. Each of the fluidic channels 720 is configured to transfer fluid through the cartridge 600. Exemplary cartridges are described in, for example, PCT Application No. PCT/US2012/068725, filed Dec. 10, 2012, and U.S. Provisional Application No. 61/696,997, filed on Sep. 5, 2012, and U.S. Provisional Application No. 61/735,215, filed on Dec. 10, 2012, each of which is hereby incorporated herein by reference in its entirety.

The microfluidic device 500 includes a plurality of fluidic channels 720 therethrough. The plurality of fluidic channels 720 on the device correspond to the plurality of fluidic channels 720 on the cartridge 600 such that, when connected, the fluidic channels 720 of the microfluidic device 500 and the cartridge 600 form one or more fluidic circuits. The fluidic circuits allow fluid communication between the microfluidic device 500 attached to the cartridge 600 and other components of the system 100.

The interconnect adaptor 300 is configured to facilitate fluidic connection between the plurality of fluidic channels 720 of the microfluidic device 500 with the plurality of fluidic channels 720 of the cartridge 600. The interconnect adaptor 300 includes a plurality of device-nozzles 340. The device-nozzles 340 form an array, and are configured to be inserted into corresponding holes 200 on the microfluidic device 500. In some embodiments, the interconnect adaptor 300 further includes a plurality of cartridge-nozzles 380. The cartridge-nozzles 380 form an array, and are configured to be inserted into corresponding holes 200 on the cartridge 600. In some embodiments, the interconnect adaptor 300 is removably connected to the cartridge 600 using, for example, cartridge-nozzles 380. In some embodiments, the interconnect adaptor 300 is a component of the cartridge 600.

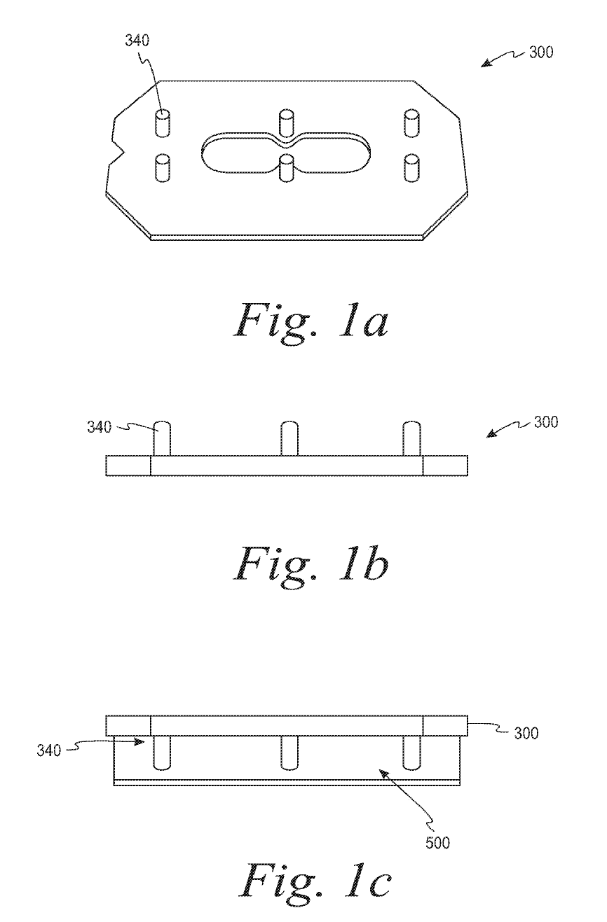

FIGS. 1A and 1B show photographs of the interconnect adaptor 300 according to some embodiments of the invention. The interconnect adaptor 300 comprises abase substrate 310 and an array of device-nozzles 340 attached to the front-side 320 of the base substrate 310.

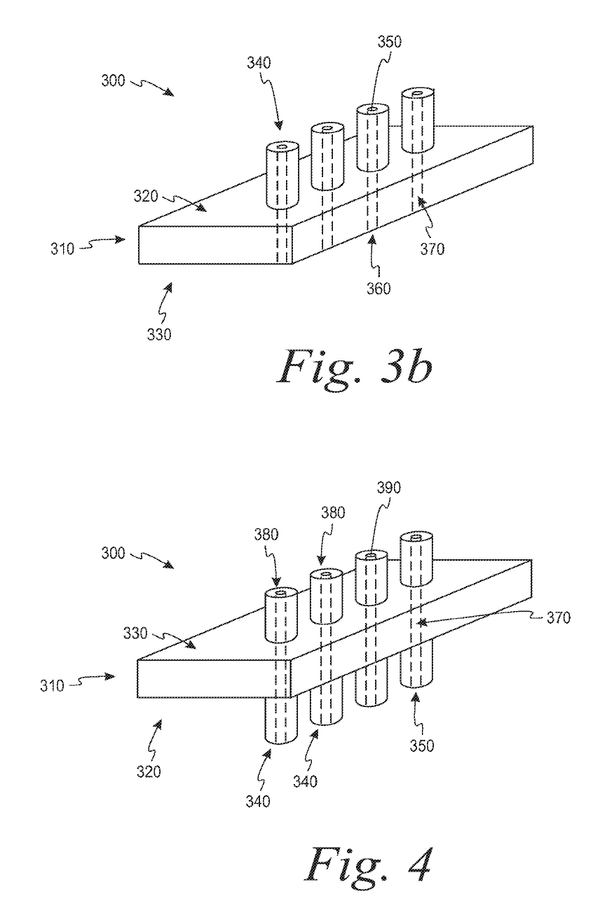

FIGS. 3A and 3B show the interconnect adaptor 300 according to some embodiments of the invention. The interconnect adaptor 300 includes a base substrate 310 having a front-side 320 and a back-side 330. FIG. 3A illustrates a perspective view of the interconnect adaptor 300 generally from the back-side 330. FIG. 3B illustrates a perspective view of the interconnect adaptor 300 generally from the front-side 320. The front-side 320 includes an array of device-nozzles 340 extending therefrom. Each device-nozzle 340 includes a device-side opening 350 connected to a respective back-side opening 360 on the back-side 330 of the interconnect adaptor 300 via a channel 370 through the base substrate 310. White the illustrated embodiment includes each device-side opening 350 connected with a respective back-side opening 360 via straight-through channel 370, it is contemplated that each device-side opening 350 can correspond with one or more back-side openings 360, that each back-side opening 360 cart correspond with one or more device-side openings 350, that the respective device-side opening 350 and back-side opening 360 may be offset from one another, combinations thereof or the like. The device-side openings 350, back-side openings 360, and the channel 370 can include one or more features to alter properties of fluid flow therethrough such as restrictions, expansions, etc.

The interconnect adaptor 300 can be attached to the cartridge 600 in a number of ways. For example, the back-side 330 of the interconnect adaptor 300 can be attached to a surface of the cartridge 600. In some embodiments, the base substrate 310 is fastened to the cartridge 600 by known methods such as screws, bolts, pins, clamps, etc. In some embodiments, the base substrate 310 is bonded with the cartridge 600 by known methods including ultrasonic welding, adhesives such as double-sided tape (e.g., 300LSE, available from 3M, St. Paul, Minn.), solvent bonding, etc. Accordingly, in some embodiments, the interconnect adaptor 300 can include an adhesive layer disposed on surface of the back-side 330. Furthermore, in some embodiments, as will be described in more detail below, the interconnect adaptor 300 can be a part of, built into, or integrally formed with the cartridge 600.

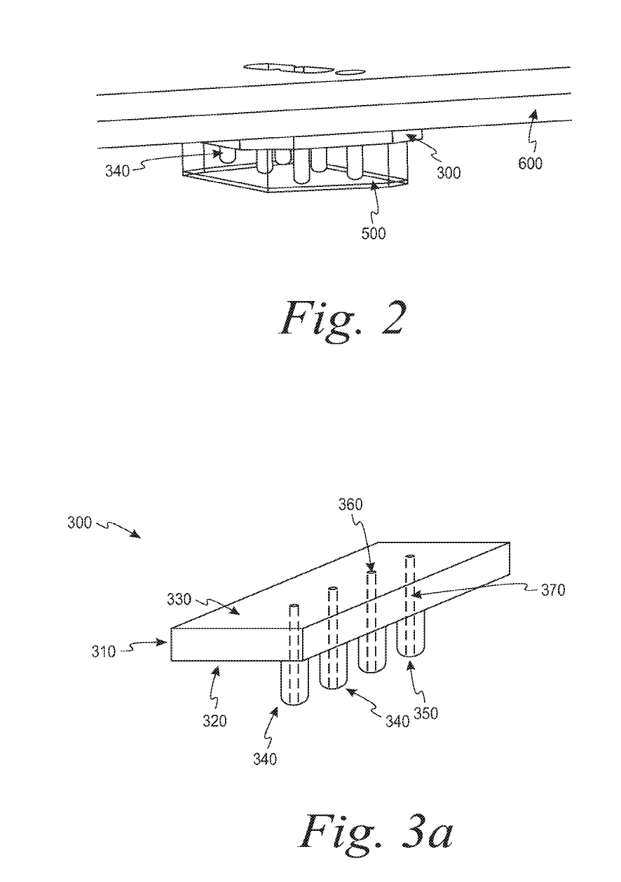

FIG. 2 shows a diagrammatic view of a microfluidic device 500 attached to a cartridge 600 via the interconnect adaptor 300. The back-side 330 of the interconnect adaptor 300 can be disposed on a surface of the cartridge 600. The nozzles on the front-side of the interconnect adaptor can be inserted into the inlets/outlets of the microfluidic device 500. The microfluidic channels in the microfluidic device 500 can be connected to the channels in the cartridge via (he through-holes in the nozzles 380.

According to some embodiments of the invention, the interconnect adaptor 300 can be fabricated as part of the cartridge 600. In one such embodiment, a surface of the cartridge 600 includes the array of nozzles that can be used for connecting with the microfluidic device 500. The cartridge 600 in this embodiment can also be the base substrate 310. The nozzles can be inserted, built, machined or formed into the cartridge. For example, the nozzles can be made at least in part by an injection-molding step that creates the cartridge or a portion thereof.

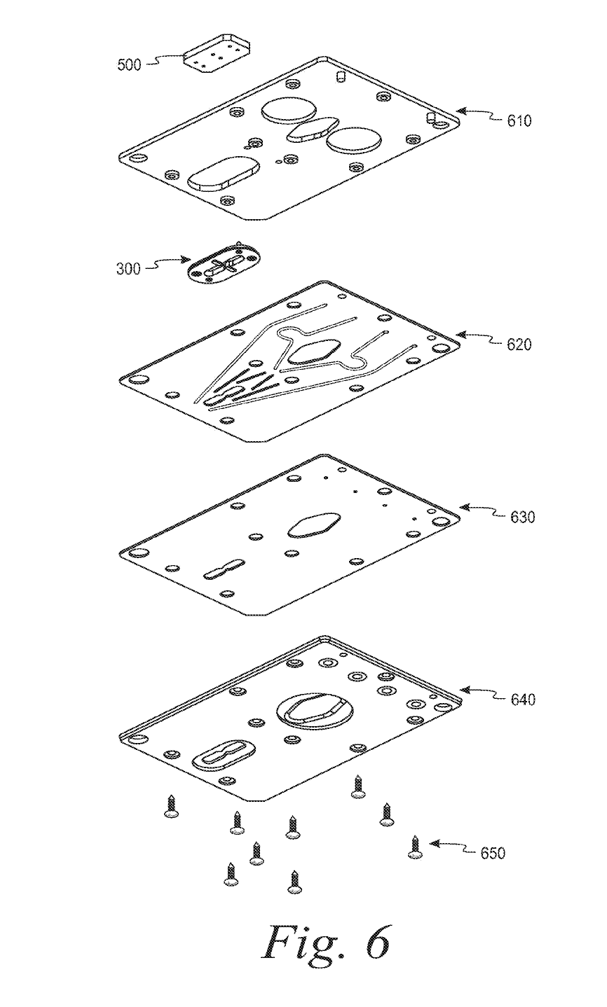

FIG. 5 shows a diagrammatic view of the interconnect adaptor 300 where the interconnect adaptor 300 is part of the cartridge 700, according to some embodiments. As shown, the cartridge 700 includes a base substrate 710 having at least one or more fluidic channels 720 disposed therein. The cartridge base substrate 710 can include a top substrate 730 and a bottom substrate 740 enclosing at least one or more fluidic channels 720. The top substrate 730 can correspond to base substrate 310 of the interconnect adaptor 300. The base substrate 310 can include an array of device-nozzles 340 on the front-side 320 of the base substrate 310. Each nozzle 340 includes a device-side opening 350 connected to a fluidic channel 720 of the cartridge 700 via a channel 370.

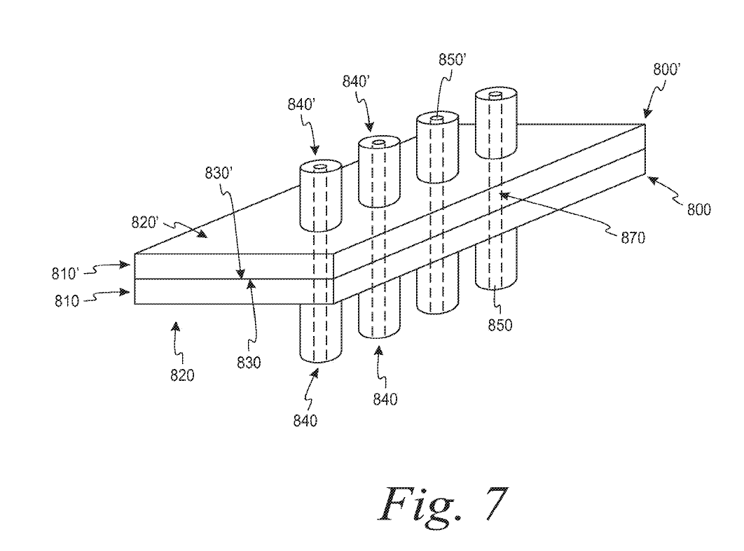

In some embodiments, the interconnect adaptor 300 can be “captured” by the cartridge 600. In one embodiment a lip on the cartridge 600 captures the separate interconnect adaptor 300 between an elastomer and another hard surface. FIG. 6 is an exploded view of one method for “capturing” the interconnect adaptor within the cartridge 600. As shown, the cartridge 600 can comprise a lower molded layer 610, a lower elastomer layer 620, an upper elastomer layer 630, and an upper molded layer 640, which can be fastened together by screws 650. The interconnect adaptor 300, for connecting the microfluidic device 500 (e.g., organ-chip), can be sandwiched between the lower molded layer 610 and the lower elastomer layer 620.

In some embodiments, the interconnect adaptor 300 includes one or more alignment features on the front-side 320 and/or back-side 330 of the base substrate 310 that aid alignment of the interconnect adaptor 300 with, for example, the cartridge 600 or the microfluidic device 500. The features can be selected from posts, ridges, notches, holes, guides, and the like. These features can also be used to uniquely identify the interconnect adaptor and its corresponding microfluidic device and/or the corresponding cartridge. Beneficially, these alignment features can be used to ensure microfluidic devices and/or cartridges of different designs are connected with their appropriate counterpart devices, and ensure the devices and cartridges are used within design parameters, such as within a predetermined pressure regime. For example, in some embodiments, the interconnect adaptor 300 includes alignment features that are configured to allow interconnect between a lower-pressure microfluidic device and lower-pressure cartridge, but win inhibit connection of a lower-pressure microfluidic device with a higher-pressure cartridge. Beneficially, this prevents damage to components of the system.

Beneficially, the nozzle array, such as cartridge-nozzles 380, can provide an alignment feature. For example, the nozzles of the nozzle array can be positioned in various locations on the surface to form unique array configurations. These unique array configurations can be used in a lock-and-key configuration with the holes 200 of a microfluidic device and/or cartridge to provide safety and testing benefits. For example, a high-pressure system can have one array configuration, and a low-pressure system can have a second array configuration so that components of the low-pressure system cannot be attached to components of the high-pressure system. Additionally, the lock-and-key configurations and/or alignment features can be used to ensure the proper orientation and/or positioning of the microfluidic device 500 and/or cartridge 600.

FIG. 1C shows a photograph of the interconnect adaptor connected with a microfluidic device. The device-nozzles 340 insert into inlets/outlets (not labeled) of the microfluidic device 500.

In some embodiments, the interconnect adaptor 300 includes one or more features on the front-side 320 and/or back-side 310 of the base substrate 310 that aid in providing a fluidic seal between the interconnect adaptor 300 and the cartridge 600.

Features such as ridges on the back-side of the interconnect adaptor can also be used to route fluid from one nozzle location to a location on the cartridge that is not concentric to the nozzle. For example, the back-side of the base substrate can form one-half of a fluid channel and the cartridge surface it mates with providing the other half of the fluid channel. This can also be achieved with a channel on the cartridge.

In some embodiments, the nozzles 340 of the interconnect adapter 300 are inserted into holes 200 (e.g. inlet/outlet ports) of the microfluidic device 500 to form a connection therebetween. The nozzles 340 can be slightly oversized so that the microfluidic device holes 200 radially compress around the nozzle, thereby forming an interference or compression fit that ensures a tight fluid connection. The radial compression creates a substantial frictional force that must be overcome to insert the nozzles into the microfluidic device. Beneficially, the radial compression force must be overcome to remove the nozzles from the microfluidic device, and, thus, can hold the microfluidic device in place during use without additional fast-nee. In some embodiments, the nozzle is formed with a diameter that is in the range of about 20% to about 50% larger than the diameter of the inlet/outlet that it is to be inserted into. In some embodiments, the nozzle is formed with a diameter that is in the range of about 10% to about 20% larger than the diameter of the inlet/outlet that it is to be inserted into. In some embodiments, the nozzle is formed with a diameter that is in the range of about 2% to about 10% larger than the diameter of the inlet/outlet that it is to be inserted into.

Additionally or alternatively, the nozzle 340 can include a connection feature to increase radial compression and improve robustness of fluid sealing. In some embodiments, the connection feature includes a barbed shape or a raised ridge that extends generally about the outer circumference.

Beneficially, the interconnect adapter 300 provides for numerous connections can be made simultaneously by pushing the microfluidic device against the interconnect adaptor nozzles. This allows for a practitioner to more easily connect microfluidic devices and cartridges as all connections are securely formed simultaneously, rather than having to ensure each of the plurality of individual connections is secure. Beneficially, the interconnect adapter 300 also provides tactile feedback for when the numerous connections are secured and fluid-tight.

According to some embodiments of the invention, the connection to a cartridge can be made utilizing the nozzles on the back-side of the base substrate. The nozzles can be inserted into holes 200 that form the inlet/outlet ports of the cartridge. The nozzles can be slightly oversized so that the cartridge inlets/outlets can radially compress around the nozzle, thereby ensuring a tight fluid connection. In some embodiments, the nozzle is formed with a diameter that is in the range of about 20% to about 50% larger than the diameter of the inlet/outlet that it is to be inserted into In some embodiments, the nozzle is forrned with a diameter that is in the range of about 10% to about 20% larger than the diameter of the inlet/outlet that it is to be inserted into. In some embodiments, the nozzle is formed with a diameter that is in the range of about 2% to about 10% larger than the diameter of the inlet/outlet that it is to be inserted into. Alternatively, the nozzles can be smaller than the holes 200 and glued in place.

The base substrate and/or the nozzle can be fabricated front any desirable material. For example, the base substrate and/or the nozzle can be fabricated from any biocompatible material(s). As used herein, the term “biocompatible material” refers to any polymeric material that does not deteriorate appreciably and does not induce a significant immune response or deleterious tissue reaction, for example, toxic reaction or significant irritation, over time when implanted into or placed adjacent to the biological tissue of a subject, or induce blood clotting or coagulation when it comes in contact with blood. Suitable biocompatible materials include polyimide derivatives, polyimide polymers, and polyimide copolymers, poly(ethylene glycol), polyvinyl alcohol, polyethyleneimine, and polyvinylamine, polyacrylates, polyamides, polyesters, polycarbonates, polyurethanes, polysulfones, cyclic olefin copolymers (COCs), cyclic olefin polymers (COPs), styrene-ethylene/butylene-styrene (SEBS), and polystyrenes.

In some embodiments, the base substrate and/or the nozzle can be fabricated from or include a material selected from the group consisting of styrene-ethylene/butylene-styrene copolymer, polydimethylsiloxane, polyimide, polyethylene terephthalate, polymethylmethacrylate, polyurethane, polyvinylchloride, polystyrene polysulfone, polycarbonate, polymethylpentene, polypropylene, a polyvinylidene fluoride, polysilicon, polytetrafluoroethylene, polysulfone, acrylonitrile butadiene styrene, polyacrylonitrile, polybutadiene, poly(butylene terephthalate), poly(ether sulfone), poly(ether ether ketones), poly(ethylene glycol), styrene-acrylonitrile resin, poly(trimethylene terephthalate), polyvinyl butyral, polyvinylidenedifluoride, poly(vinyl pyrrolidone), and any combination thereof.

In some embodiments, the base substrate is made of a rigid material such as metals or polymers.

In some embodiments, the nozzle can be formed from an elastomeric material such as silicone rubber, styrene-ethylene/butylene-styrene (SEBS), similar materials, and combinations thereof. In some embodiments, other materials can also be used, such as natural rubber materials, polydimethylsiloxane (PDMS), polyurethanes, natural or synthetic latex, or combinations thereof.

In some embodiments, the nozzle can be formed from a rigid material such as metals or polymers.

In some embodiments, a fluid-tight seal between two surfaces is formed when the two surfaces are biased together and at least one of the surfaces is deformable. Thus, the choice of material for the nozzle can depend on the materials of the respective microfluidic device or the cartridge. Similarly, the choice of material for the respective microfluidic device or cartridge can depend on the materials of the nozzle.

In some embodiments, the nozzle is formed from an elastomeric material, and the respective opening, port, or hole in the respective cartridge or device is formed within a rigid material. For example, if the respective microfluidic device or the cartridge is fabricated from a rigid material, the nozzle can be formed from an elastomeric material.

In some embodiments, the nozzle is formed from a rigid material, and the respective opening, port, or hole in the respective cartridge or microfluidic device is formed within an elastomeric material. For example, if the respective microfluidic device or cartridge is fabricated from an elastomeric material, the nozzle can be formed front a rigid material.

In some embodiments, both the nozzle and the respective opening, port, or hole in the respective cartridge or microfluidic device are formed within rigid materials, and at least a portion of either the nozzle or the respective opening, port, or hole includes an elastomeric coating that forms the seal. For example, if both the nozzle and the respective opening, port, or hole are formed from rigid materials, the nozzle can include an elastomeric coating on the outer surface. The elastomeric coating is of sufficient thickness to deform and form a liquid-tight seal between the nozzle and the respective opening, port, or hole. Similarly, the opening, port, or hole can include an elastomeric coating on the inner surface that is of sufficient thickness to deform and form a liquid-tight seal between the nozzle and the respective opening, port, or hole.

In some embodiments, both the nozzle and the respective opening, port, or hole in the respective cartridge or microfluidic device are formed within rigid materials, and at least a portion of each of the nozzle and the respective opening, port, or hole includes an elastomeric coating that forms the seal. For example, if both the nozzle and the respective opening, port, or hole are formed from rigid materials, the nozzle can include an elastomeric coating on the outer surface and the respective opening, port, or hole can include an elastomeric coating on the inner surface. These elastomeric coatings that is of sufficient thickness to deform and form a liquid-tight seal between the nozzle and the respective opening, port, or hole. The elastomeric coatings are of cooperatively of sufficient thickness to deform and form a liquid-tight seal between the nozzle and the respective opening, port, or hole.

In some embodiments, coatings are applied to at least one of the nozzle and the respective opening, port, or hole that decrease the frictional shearing force between the nozzle and the respective opening, port, or hole. These coatings may be the elastomeric coatings, or an additional coating.

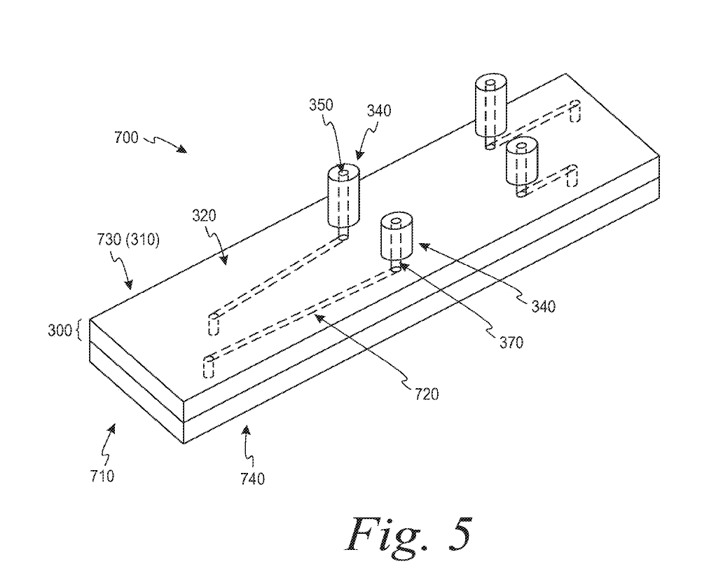

FIG. 4 shows an interconnect adaptor 300′ according to some embodiments of the invention. In the illustrated embodiment, the back-side 330 further includes an array of cartridge-nozzles 380 extending therefrom. Each cartridge-nozzle 380 corresponds to a respective device-nozzle 340, and includes a cartridge-side opening 390 connected to a respective device-side opening 350 via a channel 370 through the base substrate 310. While the illustrated embodiment includes each device-side opening 350 being connected with a respective cartridge-side opening 390 via straight-through channel 370, it is contemplated that each device-side opening 350 can correspond with one or more cartridge-side openings 390, that each cartridge-side opening 390 can correspond with one or more device-side openings 350, that the respective device-side opening 350 and cartridge-side opening 390 may be offset from one another, combinations thereof, or the like.

In some embodiments, an interconnect adaptor 300′ having device-side nozzles 340 and cartridge-side nozzles 380 is formed using two interconnect adaptors, such as interconnect adaptors 300, each having a plurality of nozzles extending front a respective front-side 320. For example, the two interconnect adaptors 300 can be manufactured separately and then the back-side 330 of the first interconnect adaptor 300 cart be bonded to the back-side 330 of the second interconnect adaptor 300 by known methods, such as ultrasonic welding, solvent bonding, gluing, etc. In some embodiments, the back-side 330 of one or both interconnect adaptors 300 includes routing channels that translate fluid between corresponding nozzles that are offset from each other. In some embodiments, each of the interconnect adaptors 300 include a plurality of back-side openings 360 that at “standardized” positions such that a variety of interconnect adaptors 300, each having different arrays of nozzles, can be bonded together in pairs to produce a larger number of unique combinations of interconnect adaptors 300′. For example, interconnect adaptors 300 having either a first array of nozzles or a second array of nozzles can be combined to create interconnect adaptors 300′ having opposing nozzle arrays in either a first-first, first-second, or second-second nozzle array pattern.

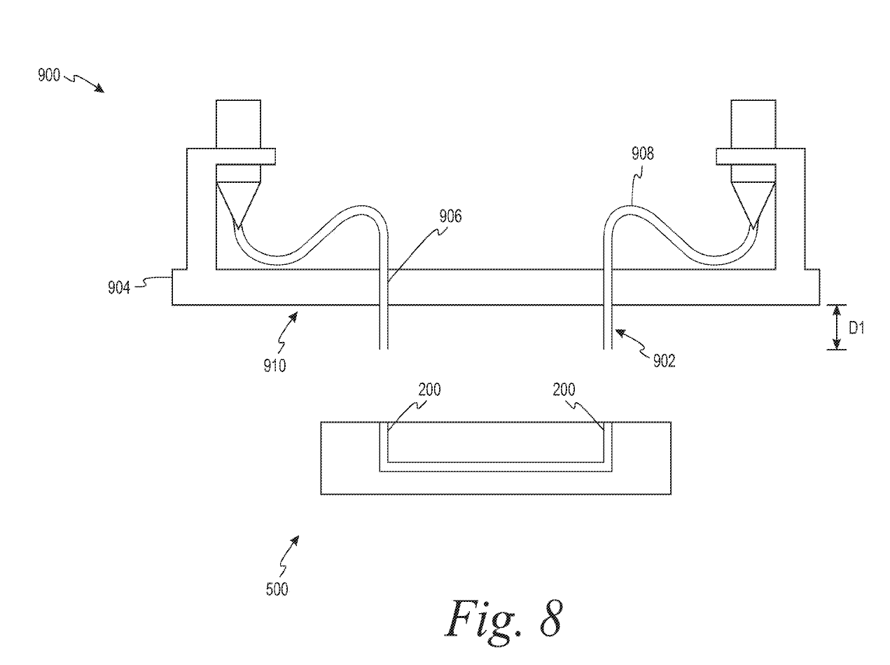

FIG. 7 schematically depicts two interconnect adaptors 800, 800′ attached together via their back-sides. The first interconnect adaptor 800 includes abuse substrate 810 having a front-side 820 and a back-side 830. The base substrate 810 includes an array of first nozzles 840 extending from its front side 820. The second interconnect adaptor 800′ includes a base substrate 810′ having a front-side 820′ and a back-side 830′. The base substrate 810′ includes an array of second nozzles 840′ extending from its front side 820′. Each first nozzle 840 includes an opening 850 which is connected to an opening 850′ of a respective second nozzle 840′ via channel 870. While channel 870 is shown as a straight-through channel, channel 870 does not need to be a straight-through channel, for example, when connected nozzles 840 and 840′ are offset from each other.

If the interconnect adaptor is to be attached to the cartridge, the back-side of the base substrate can comprise features that aid in alignment and/or fluidic seal. In some embodiments, these features can be nozzles on the back-side of the base substrate. The nozzles on the back-side of the base substrate can make interference tit with holes 200 in the cartridge to seal and hold the interconnect adaptor in place.

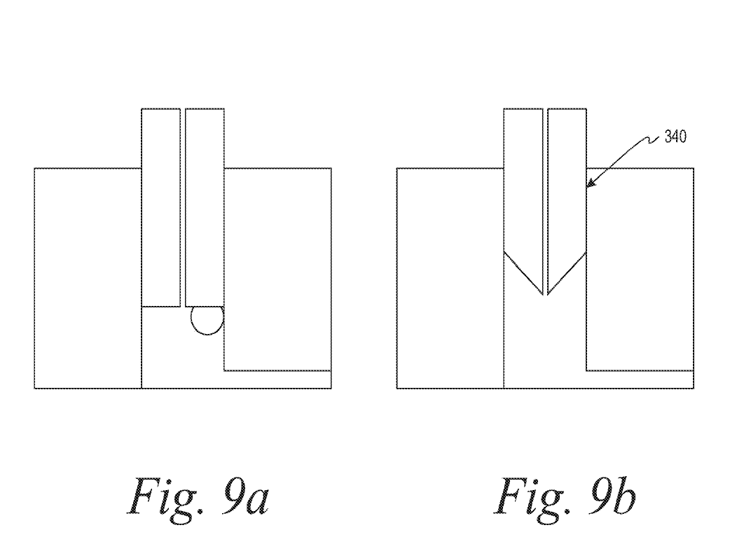

Referring now to FIG. 8, a cartridge 900 having an integrated interconnect adaptor 902 is shown. The cartridge 900 includes a substrate 904 having a plurality of apertures 906 therethrough. Each aperture 906 is configured to receive a segment of tubing 908 therethrough. Each segment of tubing 908 generally extends a predetermined distance D from a first side 910 of the cartridge substrate 904, forming a nozzle array. The tubing 908 is held in place within the aperture through, for example, a friction fit, clamp, or other known mechanism. Beneficially, the tubing 908 can plug directly into microfluidic devices or associated gaskets, greatly simplifying construction and reducing cost of cartridges and interconnect adaptors. In some embodiments, the segments of tubing 908 extend more than one distance. For example, at least one segment of tubing extends a first distance from the substrate, and at least one segment of tubing extends a second distance front the substrate.

The predetermined distances (for example D1) are selected such that the segments of tubing 908 are rigid enough to be simultaneously inserted into holes 200 (e.g., inlet/outlet ports) of the microfluidic device 500 without the need fur additional or intervening mechanisms. Selection of the predetermined distances (for example D1) is generally based on, for example, the resilience of the tubing 908, the elasticity of the microfluidic device 500, the resistive force needed to fully insert the tubing 908 into the microfluidic device 500, combinations thereof, and the like. The resilience of the tubing 908 is affected by, for example, the tubing material, inside diameter, outside diameter, etc.

The nozzles 340 can include any shape. In some embodiments, the nozzles 340 are generally cylindrically shaped. In some embodiments, the nozzles 340 are generally conically shaped. In some embodiments, nozzle characteristics are used to, for example, form a lock-and-key configuration between the interconnect adapter 300 and the cartridge 600 or microfluidic device 500. These characteristics can include, for example, shapes, sizes, resilience, sealing features, orientation relative to a surface, and the like, or combinations thereof in some embodiments, a first interconnect adapter 300 includes nozzles that all share a first characteristic, while a second interconnect adapter 300 includes nozzles all share a second characteristic. For example, in some embodiments, the first interconnect adapter 300 includes cylindrical nozzles, while the second interconnect adapter 300 includes frustoconical nozzles. In some embodiments, one or more nozzles 340 in the nozzle array have a first characteristic, while one or more nozzles 340 of the array have a second characteristic. In some embodiments, one or more cylindrical nozzles 340 have a diameter that is larger than the diameter of one or more other cylindrical nozzles 340. In some embodiments, one or more of the nozzles 340 have a length that is longer than the length of one or more other nozzles 340. In some embodiments, one or more of the nozzles 340 extend away from the surface at a different orientation than one or more other nozzles 340.

Similarly, the tips of the nozzles 340 can include any shape. In some embodiments, the tips are squared or “blunt” ends. In some embodiments, the tips are rounded. In some embodiments, the tips include tapered sides forming a frustoconical or “sharpened” tip. Beneficially, it is believed that tapered tips can ease alignment with and insertion into the inlets/outlets of the microfluidic device 500 or the cartridge.

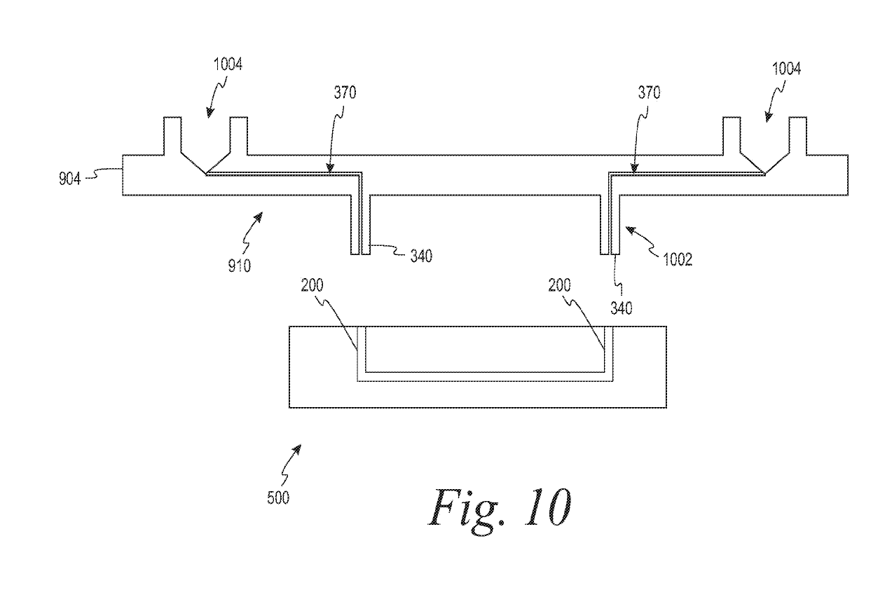

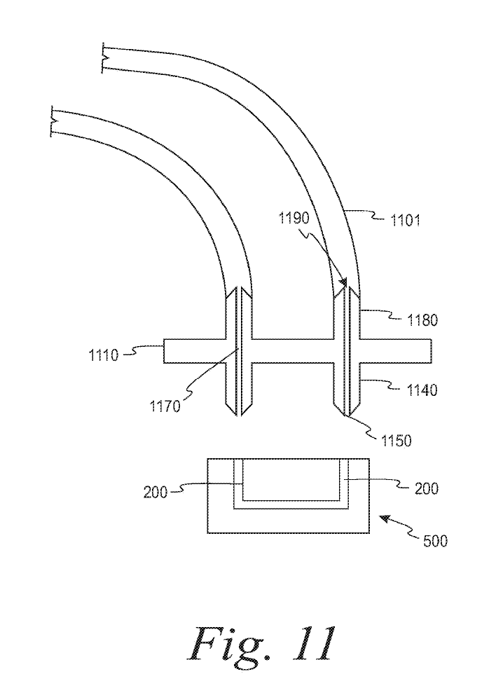

As shown in FIG. 9A, a bubble may accumulate or get trapped at a nozzle interface such as the nozzle-to-chip interface for some devices of the present disclosure. This accumulation may lower performance of the device, for example, by increasing fluidic resistance, or by dislodging and entering the cell-culture area. In some embodiments, this trapping or accumulation is reduced using a “sharpened” tip, for example, a cone. One example of a sharpened tip is shown in FIG. 9B. Surprisingly, this sharpened tip reduces bubble trapping or accumulation at the port as compared to a blunt tip despite increasing both the hydrophobic surface area of the tip and the volume for the bubbles to become trapped. This surprising result is more even more pronounced at an inlet. The nozzle 340 can either be manufactured with conical or sharpened tip or processed to provide such shapes after manufacture.

In some embodiments, the trapping or accumulation of a bubble is reduced using hydrophilic surfaces. These surfaces are less likely to trap or accumulate a bubble because they prefer to remain wetted by the aqueous liquid. The hydrophilic surface can be formed, for example, by forming the nozzles from hydrophilic materials. Examples of hydrophilic materials that can be used are: glass, certain grades of polystyrene, polypropylene, or acrylic. Additionally or alternatively, the nozzles can be treated to make them hydrophilic, for example, using a coatings, plasma treatment, etc.

Referring now to FIG. 10, a cartridge 900 having an integrated interconnect adaptor 1002 is shown. The cartridge 900 includes a substrate 904 having device-nozzles 340 and reservoirs 1004. The device-nozzles 340 extend from the base substrate 904, forming a nozzle array. The device-nozzles 340 are formed from the same material as the substrate 340. In some embodiments, the device-nozzles 340 and base substrate are polymeric materials formed, for example, using molding or 3-D printing. The reservoirs 1004 are connected to one or more respective device-nozzles 340 using fluid channels 370. When coupled to a microfluidic device 500, a fluidic circuit is formed such that fluid can travel from one reservoir 1004 to another reservoir 1004 through the microfluidic device 500.

Referring now to FIG. 11, an interconnect adaptor 1100 is shown that does not require a separate cartridge. The interconnect adaptor 1100 includes an array of device-nozzles 1140 and system-nozzles 1180 extending therefrom. Each system-nozzle 1180 corresponds to a respective device-nozzle 1140, and includes a system-side opening 1190 connected to a respective device-side opening 1150 via a channel 1170 through the base substrate 1110. The system-nozzles 1180 are coupled to the fluidic system using, for example, tubing 1101. Design considerations and properties of system-nozzles 1180 that connect to fluidic systems are similar to those considerations and properties used for nozzles that connect to microfluidic systems.

The nozzles can have different topology for different organ-chips, but can snap into generic cartridge by routing fluid to standard cartridge by internal channels. The method can be broadly generalized to many microfluidic devices, even non-elastic ones.

In some embodiments, the device-nozzles and the system-nozzles can extend from the same side of the interconnect adaptor. Moreover, in some embodiments, the cartridge is a microfluidic device.

In some embodiments of the invention, the microfluidic device is an organ-chip. As used herein, the term “organ-chip” refers to a microfluidic device which mimics at least one physiological function of at least one mammalian (e.g., human) organ. While the organ-chips are discussed herein as mimicking a physiological function of a mammalian organ, it is to be understood that organ-chips can be designed that can mimic the functionality of any living organ from humans or other organisms e.g., animals, insects, plants). Thus, as used herein, the term organ-chip in not limited to just those that mimic a mammalian organ, but includes organ-chips which can mimic the functionality of any living organ from any organism including mammals, non-mammals, insects, and plants. As such, the systems, devices, and instruments described herein can be used to model or study mammalian as well as non-mammalian (e.g., insects, plants, etc . . . ) organs and physiological systems and effect of active agents on such organs and physiological systems.

In some embodiments where the organ-chips mimic physiological functions of more than one mammalian e.g., human) organ, the organ-chips can include individual sub-units, each of which can mimic physiological function of one specific mammalian (e.g., human) organ.

Organ-chips are also referred to as organ-chip Mimic Devices or organ-on-a-chip in the art. Generally, the organ-chips comprise a substrate and at least one (e.g., one, two, three, four, six, seven, eight, nine, ten, or more) microfluidic channels disposed therein. The number and dimension of channels in an organ-chip can vary depending on the design, dimension and/or function of the organ-chip. In some embodiments, an organ-chip can comprise at least one (e.g., one, two, three, four, six, seven, eight, nine, ten, or more) microfluidic channels for the purpose of replenishing nutrients to the biological material contained within the organ-chip. An at least partially porous and at least partially flexible membrane is positioned along a plane within at least one of the channels, wherein the membrane is configured to separate said channel to form two sub-channels, wherein one side of the membrane can be seeded with vascular endothelial cells, and the other side of the membrane can be seeded with at least one type of organ-specific parenchymal cells.

Exemplary organ-chips amenable to the present disclosure are described, for example, in U.S. Provisional Application No. 61/470,987, filed Apr. 1, 2011; No. 61/492,609, filed Jun. 2, 2011; No. 61/447,540, filed Feb. 28, 2011; No. 6/449,925, filed Mar. 7, 2011; and No. 61/569,029, filed on Dec. 9, 2011, in U.S. patent application Ser. No. 13/054,095, filed Jul. 16, 2008, and in International Application No. PCT/US2009/050830, filed Jul. 16, 2009 and PCT/US2010/021195, filed Jan. 15, 2010, content of all of which is incorporated herein by reference in their entirety. Muscle Organ-chips are described, for example, in U.S. Provisional Patent Application Ser. No. 61/569,028, filed on Dec. 9, 2011, U.S. Provisional Patent Application Ser. No. 61/697,121, filed on Sep. 5, 2012, and PCT patent application titled “Muscle Chips and Methods of Use Thereof” filed on Dec. 10, 2012 and which claims priority to the U.S. provisional application Nos. 61/569,028, filed on Dec. 9, 2011, U.S. Provisional Patent Application Ser. No. 61/697,121, the entire contents of all of which are incorporated herein by reference.

The organ-chips can also have control ports for application of mechanical deformation (e.g., side chambers to apply cyclic vacuum, as in the Lung Chip described in the PCT Application No.: PCT/US2009/050830) and electrical connections (e.g., for electrophysiological analysis of muscle and nerve conduction). A similar approach of producing the Lung Chips with or without aerosol delivery capabilities as described, e.g., in the PCT Application No.: PCT/US2009/050830 and U.S. Provisional Application Nos. 61/483,837 and 61/541,876, the contents of which are incorporated herein by reference in their entirety, can be extended to produce other organ-chips, e.g., heart chips and liver chips.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects described herein, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages can mean, for example, ±1%.

The singular terms “a,” “an,” and “the” include plural referents unless context clearly indicates otherwise. Similarly, the word “or” is intended to include “and” unless the context clearly indicates otherwise. Thus fix example, references to “the method” includes one or more methods, and/or steps of the type described herein and/or which will become apparent to those persons skilled in the art upon reading this disclosure and so forth.

Although methods and materials similar or equivalent to those described herein can be used in the practice or testing of this disclosure, suitable methods and materials are described herein. The term “comprises” means “includes.” The abbreviation, “e.g.” is derived from the Latin exempli gratia, and is used herein to indicate a non-limiting example. Thus, the abbreviation “e.g.” is synonymous with the term “fix example.”

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the invention. It is also contemplated that additional embodiments according to aspects of the present invention may combine any number of features from any of the embodiments described herein.

1. An interconnect adaptor for connecting a microfluidic device to a fluidic system, the interconnect adaptor comprising:
a base substrate having a first side; and
a nozzle array including two or more nozzles, the nozzle array being located on the first side of the base substrate, the two or more nozzles extending away from the base substrate, each of the nozzles including an opening with a channel extending therefrom, the channels being configured to transport fluid between the microfluidic device and the fluidic system, each of the nozzles being configured for insertion into a respective hole in the microfluidic device, the insertion forming a radially sealed connection between each nozzle and the respective hole in response to the nozzles being inserted into the respective holes.

2. The interconnect adaptor of claim 1, wherein the base substrate is comprised of a rigid material.

3. The interconnect adaptor of claim 1, wherein the fluidic system is a cartridge containing a plurality of cartridge fluid channels, and a second side of the base substrate is disposed on a surface of the cartridge.

4. The interconnect adaptor of claim 3, wherein a second side of the base substrate is bonded with the surface of the cartridge.

5. The interconnect adaptor of claim 3 wherein the base substrate is a part of the cartridge.

6. The interconnect adaptor of claim 1, wherein the interconnect adaptor further comprises a second nozzle array including two or more nozzles, the second nozzle array being located on a second side of the base substrate, the two or more nozzles of the second nozzle array extending away from the base substrate, each of the nozzles of the second nozzle array including a second opening operatively coupled to the openings of the first nozzle array, each of the nozzles of the second nozzle array being configured to be inserted into a respective hole in the fluidic system.

7. The interconnect adaptor of claim 1, wherein each nozzle has an outer diameter that is greater than a greatest dimension of the respective hole of the microfluidic device.

8. The interconnect adaptor of claim 1, wherein the nozzles are comprised of an elastomeric material.

9. The interconnect adaptor of claim 1, wherein one of the nozzles serves as an inlet and delivers the fluid to the microfluidic device, and another of the nozzles serves as an outlet and receives the fluid from the microfluidic device.

10. The interconnect adaptor of claim 1, further comprising at least one alignment feature on the first side or on a second side opposing the first side.

11. The interconnect adaptor of claim 1, wherein the nozzles include end portions that are tapered to reduce the accumulation of bubbles.

12. The interconnect adaptor of claim 1, wherein the nozzle array include nozzles forming a lock-and-key arrangement such that the nozzles can be inserted into the respective holes of only certain microfluidic devices that satisfy a predetermined criterion.

13. The interconnect adaptor of claim 1, wherein said microfluidic device is an organ-chip having a porous membrane with cells on at least one surface of the porous membrane, the transport fluid from at least one nozzle of the interconnect adaptor provides nutrients to the cells.

14. An interconnect adaptor for connecting a fluidic system to a compatible microfluidic device, the interconnect adaptor comprising:
a base substrate having a first side; and
a nozzle array including two or more nozzles, the nozzle array being located on the first side of the base substrate, the two or more nozzles extending away from the base substrate, each of the nozzles including an opening with a channel extending therefrom, the channels being configured to transport fluid between the compatible microfluidic device and the fluidic system, each of the nozzles being configured to be inserted into a respective hole in the compatible microfluidic device, the nozzles of the nozzle array forming a lock-and-key arrangement such that the nozzles can be inserted into the respective holes of only microfluidic devices that satisfy a predetermined criterion, the lock-and-key arrangement including at least first nozzle having a first characteristic and at least second nozzle having a second characteristic, the second characteristic being different from the first characteristic.

15-16. (canceled)

17. The interconnect adaptor of claim 14, wherein the first and second characteristics are associated with one of the group consisting of different shapes, different sizes, different resilience, different sealing features, and different orientation relative to a surface.

18. The interconnect adaptor of claim 14, wherein the predetermined criterion is based on flow rate of the microfluidic device.

19. The interconnect adaptor of claim 14, wherein the predetermined criterion is based on pressure of the microfluidic device.

20. The interconnect adaptor of claim 14, wherein the predetermined criterion is based on the functionality of the microfluidic device.

21. The interconnect adaptor of claim 20, wherein the microfluidic device is an organ chip having a porous membrane with cells on at least one surface of the porous membrane, the transport fluid from at least one nozzle of the interconnect adaptor provides nutrients to the cells.

22. A microfluidic system for connection to a fluidic system, comprising:
an interconnect adaptor including a base substrate and a nozzle array extending away from a first side of the base substrate, the nozzle array including a plurality of nozzles, each of the nozzles including a fluid channel, the fluid channels being configured to transport fluid associated with the fluidic system, the nozzle array including an inlet nozzle for transporting the fluid from the fluidic system and an outlet nozzle for transporting the fluid back to the fluidic system; and
a microfluidic device including a microchannel at least partially defined by a porous membrane having cells on at least one surface thereof, each of the nozzles being inserted into a respective hole in the microfluidic device, the inlet nozzle being inserted into an inlet hole in the microfluidic device and delivering the fluid to the cells within the microchannel, the outlet nozzle being inserted into an outlet hole in the microfluidic device and receiving the fluid from the microchannel, the inlet and outlet nozzles forming a sealed connection with the inlet and outlet holes, respectively, in response to the insertion of the inlet and outlet nozzles.
